(19) United States
(12) Patent Application Publication
(10) Pub. No.: US 2008/0306602 A1
(43) Pub. Date: Dec. 11, 2008

(54) Tibia Cement Impaction System

(76) Inventors: Richard L. Worland, Richmond, VA (US); Jonathan Keenan, Ivybridge (GB)

Correspondence Address: LAUBSCHER & LAUBSCHER, P.C., 1160 SPA ROAD, SUITE 2B, ANNAPOLIS, MD, 21403, US

(21) Appl. No.: 11/759,325

(22) Filed: Jun. 7, 2007

(51) Int. Cl. A61F 2/38

(52) U.S. Cl. 623/20.14; 623/20.32

(57) ABSTRACT

A tibial cement impaction system and method are characterized by disposable frame and plunger elements which are used to drive bone cement under pressure into apertures formed in the top surface of the tibia to prevent a subsequently applied tibia plate or component of a knee prosthesis from loosening from the tibia. To prepare the tibia, the surgeon removes the top damaged surface of the tibia to define a relatively flat surface and drills a plurality of small holes or apertures into the surface. The holes are spaced across the surface. Next, the surgeon arranges the frame over the prepared tibia surface. The frame includes a wall having a generally kidney-shaped configuration which matches the outer perimeter of the top portion of the tibia and defines a chamber. Bone cement is arranged in the chamber and then the plunger, which is configured to match the contour of the wall, is pressed into the chamber, forcing the bone cement into the apertures and evenly across the top surface of the tibia. The plunger and frame are removed and discarded. Next, the tibia plate is arranged on the bone cement and driven against the tibia with the stem portion of the plate extending through the cement into the tibia. When the cement hardens, the plate is securely connected with the tibia.

### BACKGROUND OF THE INVENTION

The knee joint is formed by three articulating bones. One bone is the patella (the knee cap), the second bone is the femur, and the third bone is the tibia. Articulating cartilage on the surfaces of the femur and tibia and behind the knee cap absorbs stress and allows the knee to glide easily. As a result of injury and aging, cartilage is worn away resulting in pain and deformation of the knee. By replacing all or part of the knee with a prosthesis, the patient can function again without pain. Knee replacements are so successful that in the United States, over 400,000 replacements are performed each year.

In knee replacement surgery, a tibia plate, typically having a depending stem, is cemented onto the top of the tibia and a femoral component is connected with the lower surface of the femur. The tibia plate has a layer of smooth material (polyethylene) which forms the articulating surface in place of the cartilage in a healthy knee. With current techniques and prosthetic materials, knee replacements typically last for at least ten years.

The most common cause for failure of a knee replacement is an infection, and the second most common cause for failure is loosening of the tibial component of the prosthesis. In many patients, the cement which secures the tibia plate to the tibia becomes loose. The present invention was developed in order to provide a more secure connection between the cement and the tibia so that the plate remains secured to the tibia.

### SUMMARY OF THE INVENTION

According to the invention, a tibial cement impaction system and method are provided for applying bone cement to the top portion of a tibia during knee replacement surgery. The system includes a disposable frame formed of synthetic plastic material having a handle at one end and a peripheral wall connected to the handle. The wall is configured to match the horizontal cross-section of the top of the tibia and defines a hollow through-chamber. The system further includes a disposable plunger having an outer surface which is configured to match the configuration of the inner surface of the frame wall so that the plunger fits snugly within the chamber. The plunger is also formed of synthetic plastic material and includes a handle. The frame is positioned on top of the tibia and bone cement is inserted into the chamber. The plunger is then inserted into the chamber and forced against the cement which in turn forces the cement into uniform contact with and into the top surface of the tibia. The plunger and frame are then removed and a tibia plate is placed on the cement. When the cement cures, the plate is secured to the tibia.

Preferably, the top surface of the tibia contains a plurality of small spaced holes which are drilled therein prior to the application of bone cement. As the plunger is forced into the chamber of the frame, the bone cement is impacted into the holes and into the softer bone in the center of the tibia to provide a more secure connection of the plate with the tibia. In addition, a larger hole is punched or drilled into the tibia top surface for receiving bone cement and then subsequently receiving the stem depending from the bottom surface of the tibia plate. As the stem enters the hole, excess cement is forced out of the hole, with the cement enveloping the stem and abutting against the lower surface of the plate.

The method for connecting a tibia plate with the tibia of a patient according to the invention includes forming a plurality of spaced apertures in the top surface of the tibia and a larger opening in the central portion of the tibia. A wall is arranged around the perimeter of the tibia upper surface to define a chamber above the surface. Bone cement is inserted into the chamber, filling the chamber to approximately three-fourths of its capacity. The bone cement is then forced into the apertures and opening and across the surface of the tibia via a disposable impaction system. The wall is then removed from the tibia, and the tibia plate is arranged on the bone cement with the stem extending into the opening. As the cement cures, the plate is secured to the tibia.

The tibia impaction system includes a frame for the wall and a plunger which is configured to match the configuration of the wall so that when the plunger is pressed into the chamber, it fits snugly against the wall to force the bone cement into the apertures. The apertures are preferably drilled into the top surface of the tibia, and the central opening is either drilled or punched into the tibia top surface.

### BRIEF DESCRIPTION OF THE FIGURES

Other objects and advantages of the invention will become apparent from a study of the following specification when viewed in the light of the accompanying drawing, in which:

FIG. 1 is a perspective view of the frame of the impaction system according to the invention;

FIG. 2 is a perspective view of the plunger of the impaction system according to the invention;

FIG. 3 is a perspective view of the impaction system arranged above a tibia upper surface prior to application of bone cement according to a method of the invention; and

FIG. 4 is a perspective view showing the connection of a tibia plate with a tibia according to the method of the invention.

### DETAILED DESCRIPTION

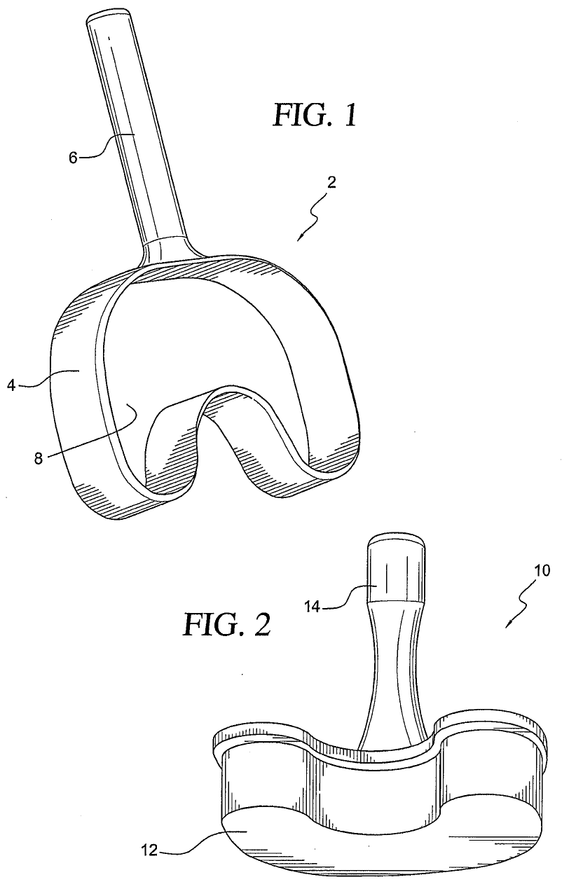

Referring first to FIGS. 1 and 2, the tibia cement impaction system according to the invention will be described. In FIG. 1 is shown a frame 2 having a wall portion 4 and a handle portion 6 extending from the wall portion. The wall portion has a kidney-shaped configuration and is designed to match the configuration of a cross-section at the top of the tibia bone. The wall portion defines a through chamber 8 for receiving bone cement as will be developed in greater detail below.

The second component of the impaction system of the invention is a plunger 10 as shown in FIG. 2. The plunger includes a solid lower portion 12 having a handle portion 14 extending from the top of the lower portion. The plunger lower portion also has a kidney-shaped configuration so that it fits snugly within the through chamber of the frame.

Both the frame and the plunger are formed of an inexpensive rigid material such as synthetic plastic which is molded into the desired configuration. The frame and plunger can be provided in any size, so that differently sized systems can be provided to accommodate different tibia sizes. The impaction system is disposable. Accordingly, the system can be sold as a unit in sterile packaging, used during knee replacement surgery on a particular patient, and then discarded.

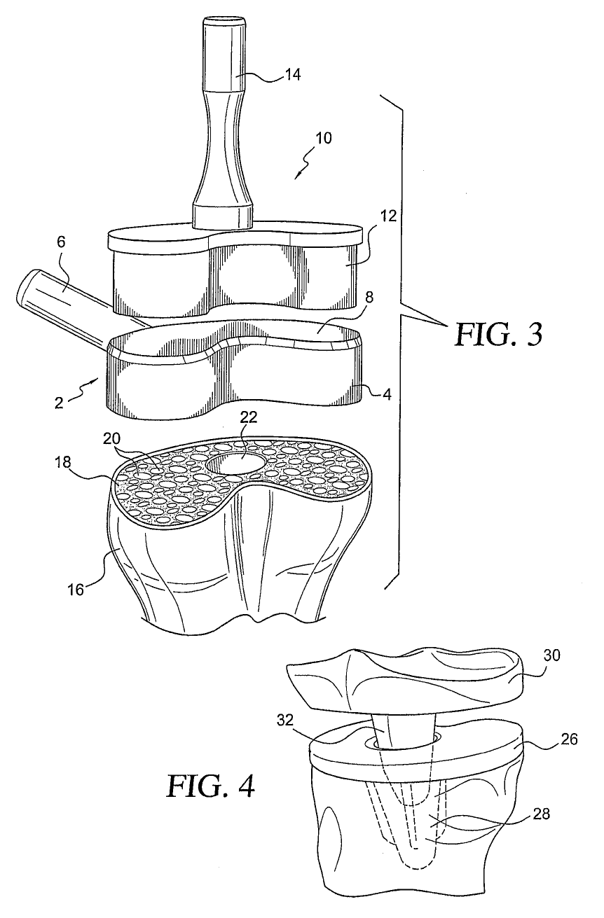

Referring now to FIG. 3, the operation of the impaction system will be described together with the method for impacting tibia cement during knee replacement surgery according to the invention. The surgeon first prepares the tibia by cutting or sawing off the damaged upper portion of the tibia 16 to define a relatively flat upper surface 18. A plurality of apertures 20 are drilled into the top surface in both the harder portion of the bone closer to the outer surface as well as into the softer portion of the bone toward the center. In addition, a central opening 22 is drilled or punched into the central opening. The location of the central opening is determined by using a template or guide (not shown) which corresponds to the location of the stem depending from the tibia plate of the knee prosthesis as is known in the art.

Once the tibia surface is prepared, the surgeon selects the appropriate size impaction system and arranges the frame 2 of the selected system on the top of the tibia 16, with the wall extending around the perimeter of the tibia top surface 18. The kidney-shaped wall matches the configuration of the tibia perimeter. Next, bone cement is placed in the through chamber 8 within the wall 4 of the frame. Preferably, the chamber is at least half filled to three quarters filled with cement. The lower portion 12 of the plunger 10 is then arranged in the chamber and forced under pressure against the cement. If desired, a mallet or hammer can be used to strike the end of the plunder handle 14 to further force the plunger against the cement. The pressure on the bone cement forces it into the apertures 20 in the upper surface of the tibia as well as into the opening 22. In addition, cement is forced into the softer portion of the bone and evenly distributed across the top surface 18 of the tibia.

The plunger and frame are then removed and the tibia plate or component 24 of the knee prosthesis is installed as will be described with reference to FIG. 4. The tibia plate 24 can have different configurations as is known in the art. The plate shown in FIG. 24 has two components, a metal plate 26 having a stem 28 depending from the bottom surface thereof and a polyethylene insert 30 also having a stem 32 which depends from the bottom of the insert and into the stem 28 of the plate 26. If desired, a portion of bone cement is applied to the stem 28 and to the bottom surface of the place 26. The stem is then inserted into the opening 22 in the tibia surface and the plate is seated on the cement layer on top of the tibia. The plate can be driven into the tibia for a secure connection. The cement is allowed to cure and the tibia plate is secured to the top of the knee. The insert can be pre-assembled with the plate or connected with the plate in a conventional manner after the plate has been implanted into the tibia. The upper surface of the insert acts in place of the cartilage of a damaged knee and cooperates with the femoral component of a knee prosthesis.

Using the impaction system and method according to the invention greatly reduces the likelihood that the cement or tibia plate will loosen from the tibia over time. By driving the cement down into the bone, excellent cement interdigitation and secure fixation of the tibial component is achieved.

Although the invention has been described in connection with full knee replacement procedure, it may also be used in a partial replacement procedure. Differently configured frames can be formed with walls which extend over only the portion of a tibia upper surface which is to be replaced. Matching plungers can also be formed. The procedure of forming apertures in the top surface of the tibia and then impacting bone cement therein using a modified impaction system is the same.

While the preferred forms and embodiments have been illustrated and described, it will be apparent to those of ordinary skill in the art that various changes and modifications may be made without deviating from the inventive concepts set forth above.

What is claimed is:

1. Apparatus for impacting bone cement into a top surface of a tibia of a patient during knee replacement surgery, comprising
(a) a disposable frame having a peripheral wall generally configured to match the horizontal cross-section of the top of the tibia, said wall defining a hollow chamber; and
(b) a disposable plunger having an outer surface which is configured to match the configuration of an inner surface of said frame peripheral wall so that said plunger fits snugly within said wall chamber, whereby when said frame is positioned on top of the tibia and bone cement is arranged in said chamber, said plunger is inserted into said chamber and forced against the cement, thereby forcing the cement into uniform contact with and into the top surface of the tibia to retain a tibia plate on the top of the tibia.

2. Apparatus as defined in claim 1, wherein the tibia is provided with a plurality of spaced apertures within the top surface thereof, said plunger forcing bone cement into said apertures to further connect the tibia plate with the tibia.

3. Apparatus as defined in claim 2, wherein said peripheral wall has a generally kidney-shaped horizontal cross-sectional configuration.

4. Apparatus as defined in claim 3, wherein said frame and said plunger each include an elongated handle connected therewith.

5. Apparatus as defined in claim 4, wherein said frame and said plunger are formed of rigid synthetic plastic material.

6. A method for connecting a tibia plate with a tibia of a patient during knee replacement surgery, comprising the steps of
(a) forming a plurality of spaced apertures in a top surface of the tibia;
(b) arranging a disposable wall above the top surface of the tibia, said wall defining a chamber above said tibia top surface;
(c) arranging bone cement within said chamber;
(d) forcing said bone cement into said apertures and across the top surface of the tibia;
(e) removing said wall from the top surface of the tibia; and
(f) arranging the tibia plate on said bone cement, whereby said cement secures the tibia plate to the top surface of the tibia.

7. A method as defined in claim 6, wherein said forcing step comprises inserting a disposable plunger having a configuration matching the configuration of said wall chamber into said chamber to impact the cement with and into the top surface of the tibia.

8. A method as defined in claim 7, and further comprising the step of preparing the tibia to form a generally flat upper surface prior to forming a plurality of spaced apertures therein.

9. A method as defined in claim 8, wherein said tibia plate includes a stem depending from a lower surface thereof, and further comprising the step of forming an opening in the top surface of the tibia for receiving the stem.

10. A method as defined in claim 6, wherein said apertures are formed in the top surface of the tibia via drilling.

11. A method as defined in claim 7, wherein said wall and said plunger are formed of rigid synthetic plastic material.

12. A method as defined in claim 11, wherein said wall has a generally kidney-shaped horizontal cross-sectional configuration which corresponds with the perimeter of the tibia top portion.
